(19) United States
(12) Patent Application Publication
(10) Pub. No.: US 2005/0170477 A1
(43) Pub. Date: Aug. 4, 2005

(54) Process for preparing conjugated linoleic acid

(76) Inventors: Marjatta Vahvaselka, Helsinki (FI); Pekka Lethnen, Veikkola (FI); Simo Laakso, Turku (FI)

Correspondence Address: NIXON & VANDERHYE, PC, 901 NORTH GLEBE ROAD, 11TH FLOOR, ARLINGTON, VA, 22203, US

(21) Appl. No.: 10/508,987

(22) PCT Filed: Mar. 27, 2003
(86) PCT No.: PCT/FI03/00236

(30) Foreign Application Priority Data
Mar. 27, 2002 (FI) 20020593

(51) Int. Cl. C12P 7/64; C12N 1/21

(52) U.S. Cl. 435/134; 435/252.3

(57) ABSTRACT

The invention relates to a process for preparing conjugated linoleic acid. In particular, the invention discloses a process for preparing conjugated linoleic acid, particularly the cis-9,trans-11 isomer thereof from grain by means of beneficial bacteria. The invention also relates to the products prepared by the process.

### FIELD OF THE INVENTION

The invention relates to a process for preparing conjugated linoleic acid. In particular, the invention discloses a process for preparing conjugated linoleic acid and in particular the cis-9,trans-11 isomer thereof from grain by means of beneficial bacteria.

### BACKGROUND OF THE INVENTION

CLA is a generic term for different isomers of conjugated linoleic acid, of which only two isomers (cis-9,trans-11 isomer, i.e. bovine acid, and trans-10,cis-12 isomer) have been found to be biologically active. Synthetic CLA products are commercially available but these usually include all different isomers of CLA and only 40% of c9,t11 isomer. Also, animal products, such as meat and milk, can be used as the CLA source. An advantage of these is that most of the CLA they contain is c9,t11 isomer, e.g. milk CLA contains 80% of c9,t11-18:2 isomer.

Several studies have shown that animal fats include a fatty acid which prevents, cancer in test animals, affects growth factors and may regulate the amount of body fat tissue. When researching hamburger steaks, Michael Pariza found that they include a fatty acid which was analyzed as conjugated linoleic acid (CLA). In studies carried out on test animals, it was found that the occurrences of breast cancer, gastric cancer and cancer of the large intestine had decreased in the group fed with food containing CLA compared to the control group (Pariza, M. W., Loretz, L. J., Storkson, J. M. and Holland, N. C. 1983. Mutagens and modulator of mutagenesis in fried ground beef. Cancer Res. (Suppl.) 43: 2444-2446, and Pariza, M. W, and Hargraves, W. A. 1985. A beef-derived mutagenesis modulator inhibits initiation of mouse epidermal tumors by 7,12-dimethylbenzanthracene. Carcinogenesis 6:591-593). Also, CLA has been able to inhibit development of cancer cells in tissue cultures of human cells. The mode of action is still unknown, but CLA has been found to have influence on the different development stages of cancer, several growth factors and possibly also on metabolism of carcinogenic substances in the liver. It has also been suggested that CLA would function as an antioxidant (Ip, C., Chin, S. F., Scimeca, J. A., and Pariza, M. W. 1991. Mammary cancer prevention by conjugated dienoic derivatives of linoleic acid. Cancer Res. 51:6118-6124), in which case the compound would protect cell membranes from the adverse effects of free radials. In addition, the decreasing effect of the compound on the cholesterol level has been studied, and it has been found that the compound does not decrease the amount of good HDL as the cholesterol decreasing pharmaceuticals do (Lee, K. N., Kritchevsky, D., and Pariza, M. W. 1994. Conjugated linoleic acid and atherosclerosis in rabbits. Atherosclerosis 108:19-25). CLA may also help weight-watchers since the compound has been found to degrade fat tissue (Park et al. 1999. Changes in Body Composition in Mice During Feeding and Withdrawal of Conjugated Linoleic Acid. Lipids 34, 243-248).

Unconjugated linoleic acid has been found to have adverse effects, such as a stimulating effect on breast cancer. The antimicrobial effect of unconjugated linoleic acid is also generally known.

CLA can be prepared chemically or enzymatically by isomerizing linoleic acid. Natural CLA is formed from i.a. multi-unsaturated fatty acids as a result of the action of the bacterium Butyrivibrio fibrisolvens in the rumen of ruminants, from which it is secreted into milk and meat, which have been found to be the best sources of CLA.

It has been noted the amount of CLA obtained from food has decreased considerably during the past decade. It has been calculated in food content analyses that in the 1970's, an average diet contained about 0.45 g of CLA/day. As the use of milk and dairy products has declined, the average intake is nowadays 0.25 g of CLA/day. Increase of the amount of natural CLA in food is very important in respect of public health since duplication of the CLA intake would, according to research, decrease the risk of cancer, for instance.

As regards food products, the importance of milk as the source of CLA has been highlighted in several studies. For example, according to a Finnish demographic study (Knekt et al., oral communication), the use of milk reduced the risk of breast cancer. Nowadays the CLA content of milk fat varies periodically to a considerable extent (2.4 - 28.1 mg/g of fat) depending on the feed quality.

It has been found that intestinal beneficial microbes form CLA. In particular, the rumen bacterium Butyrivibrio fibrisolvens and its isomerase enzyme have been studied. However, this bacterium is anaerobic to such an extent that CLA production by means of it is not feasible in industrial scale since it is difficult and uneconomical to arrange the strict anaerobic conditions required by the strain (U.S. Pat. No. 5,856,149, Pariza et al.).

It has been found that the species Propionibacterium agnes forms CLA, too, but this pathogenic strain also produces reductase enzyme, which reduces the produced CLA to other fatty acids (Verhulst et al., System. Appl. Microbiol. 9 (1987) 12-15).

It is further generally known that certain propionic acid bacteria can convert linoleic acid into its conjugated cis-9,trans-11 form. In addition, it is general knowledge that the conversion of free linoleic acid into CLA is more effective than that of triglyceride fatty acid. However, free linoleic acid has a growth inhibiting effect on propionic acid bacteria already in relatively small concentrations, which has so far prevented large scale production of conjugated linoleic acid and particularly the cis-9,t-11 form thereof.

U.S. Pat. No. 5,856,149, Pariza and Yang, describes a process for producing cis-9,trans-11 fatty acid by conversion of unconjugated unsaturated (double bonds at positions 9 and 12) fatty acid by means of the Lactobacillus reuterii strain, preferably the L. reuterii PYR8 strain. The publication describes isolation of CLA-producing strains and states that only 4 out of 45 isolated strains had the desired linoleate isomerase activity, i.e. these were able to produce CLA from linoleic acid. The publication does not mention the inhibiting effect of free linoleic acid on bacterial growth nor does it set forth a solution for avoiding this problem.

In Production of conjugated linoleic acid by daily starter cultures, J. Appl. Microbiol. 85 (1998), PP. 95-102, J. Jiang, L. Björck and R. Fonden describe the ability of propionic acid bacteria to convert linoleic acid into CLA. Having noticed that mature cheeses contain higher amounts of CLA than other dairy products, Jiang et al. studied the ability of 19 different starter bacteria to convert linoleic acid added to a culture medium into CLA. They studied the ability of 7 lactobacillus strains, 4 lactococcus strains, 2 streptococcus strains and 6 propionic acid bacteria to produce CLA from linoleic acid on MRS, milk and Na lactate culture media. In addition, different linoleic acid concentrations were studied by adding linoleic acid to MRS broth in aqueous solution of Tween 80 detergent. Only a few propionic acid bacteria out of the analyzed bacteria showed bioconversion activity; three out of six strains showed activity, i.e. Propionibacterium freudenreichii ssp. freudenreichii PFF and PFF6 and P. freudenreichii ssp. shermanii PFS. The maximal production of 265 μg/ml of CLA from an original linoleic acid concentration of 750 μg/ml was achieved with the PFF6 strain. The produced CLA contained 70 to 90% of biologically active c9,t11 isomer. None of the lactobacilli, lactococci or streptococci was found to produce CLA.

The best propionic acid bacterium, the PFF6 strain, was thus able to convert only 35% of the added linoleic acid into CLA by the technique described by Jiang et al. The researchers found that the CLA production of the propionic acid bacteria correlated positively with their tolerance with respect to free linoleic acid. Consequently, this study confirmed the generally known fact that linoleic acid has an antimicrobial effect that inhibits bacterial growth. The publication states that the effect of antimicrobial fatty acids can be reduced by using surface-active agents, such as detergents, e.g. Tween 80, or proteins. However, such studies have not been carried out and the publication does not disclose feasible, useful techniques.

WO 99/29886, J. Jiang, L. Björck and R. Fonden, is partly based on the research results described in the above-mentioned article. The application relates to the use of certain bacteria useful in food product applications in in vitro production of CLA. In addition to Propionibacterium freudenreichii ssp. freudenreichii and P. freudenreichii ssp. shermanii, Bifidobacterium breve is mentioned as a suitable bacterium. According to the publication, fermentation can be carried out in the presence of an emulsifying agent, such as Tween 80 and lecithin. The examples of this publication do not describe the use of an emulsifying agent, either, and the result given as the best result is the same as in the above-mentioned article: 246.4 μg/ml of biologically active c9,t11 isomer was obtained from an original linoleic acid concentration of 750 μg/ml using the PFF6 strain. Thus the yield was below 33%.

Finnish patent 88856 describes a process for preparing a fermented food product which contains living microorganisms and is mainly based on oat bran. Oat bran is fermented either as such or after heat treatment, and lactic acid bacteria, in particular Lactobacillus acidophilus, are used as microorganisms. The object of the invention described in the publication is to utilize the high fibre content of oat in new kind of food product. As an example the publication gives a yoghurt-type snack. The publication does not mention linoleic acid or conjugated linoleic acid produced therefrom.

Consequently, there is still a clear need for new processes for producing conjugated linoleic acid. When CLA is to be produced by means of microbiological processes, it is essential how the problems related to the toxicity and antimicrobial effect of external linoleic acid can be minimized or avoided. Processes where new raw materials can be utilized in the CLA production are also very welcome.

### BRIEF DESCRIPTION OF THE INVENTION

The present invention is based on utilization of grain as the source of linoleic acid. As regards different grain species, oat is deemed to be the preferred source of linoleic acid.

The invention thus relates to a process for preparing conjugated linoleic acid (CLA) from linoleic acid, the process utilizing grain including linoleic acid as the source of linoleic acid.

The process of the invention preferably comprises two steps where grain fat is hydrolyzed to release linoleic acid therefrom and the released linoleic acid is isomerized into conjugated linoleic acid by means of microbes.

The invention also relates to the use of grain in the preparation of conjugated linoleic acid.

The invention further relates to products prepared by the process of the invention and to their use as such or in the preparation of functional substances.

### DETAILED DESCRIPTION OF THE INVENTION

The process of the invention for preparing conjugated linoleic acid by means of a microorganism is characterized in that grain fat including linoleic acid is hydrolyzed and the linoleic acid released by hydrolysis is isomerized into conjugated linoleic acid by means of a microorganism.

The invention is thus based on the use of grain as the source of linoleic acid. According to the invention, linoleic acid is released from grain by means of a hydrolysis reaction of fat. In connection with the present invention it has been surprisingly found that when grain material is used as the source of linoleic acid as described in this application, linoleic acid does not inhibit the isomerization reaction. When grain, particularly oat, is used as the starting material, it can be ensured that linoleic acid is available to microbes for the whole duration of isomerization without the linoleic acid preventing the functioning of the bacteria.

The grain used as the source material can be any grain that includes linoleic acid and is suitable for use as the starting material of an edible product. Oat, barley, rye, wheat and malts prepared therefrom can be mentioned as examples. Suitable raw materials include untreated and treated grains and fractions prepared therefrom.

According to the present invention, oat is the most preferred starting material. The linoleic acid content of oat is about 2 to 4% of dry solids, and most of the linoleic acid is bound to diglycerides and triglycerides. Oat also contains lipase enzyme which degrades diglycerides and triglycerides into free fatty acids. Considering the objects of the invention, oat is an advantageous raw material due to its high linoleic acid content and natural lipase activity.

The natural lipase activity of grain, particularly oat, can be utilized in the hydrolysis reaction of fat. The enzyme activity required by the reaction can also be added externally. The CLA yield can be improved both in the case of oat and especially other grain by adding lipase enzyme to the reaction according to the need.

The enzymatic hydrolysis of oat fat or fat of another grain can also be facilitated by pretreatment. One advantageous pretreatment method is malting, which can be used to produce lipase activity in grain. Other suitable pretreatments include crushing, grinding, pulverizing, and dissolution in a suitable solvent, particularly in water or another liquid medium.

The lipolysis of oat, for example, can be initiated by crushing oat grains and adding water to the crushed oat. Free linoleic acid formed in the lipolysis partly binds to other components of oat, which decreases the amount of linoleic acid available to the isomerization reaction, and which should thus be avoided.

The problem can be reduced or even eliminated by the selection of suitable reaction conditions. In the selection of conditions, it is most essential to prevent the characteristic pH decrease of the oat material by keeping the pH at a sufficiently high level during the linoleic acid isomerization step. A suitable pH is 6.5 to 9, for instance. Preferably, the pH is adjusted to a level of about 7.0 to about 9.0, preferably to a level of about 8.0 to about 8.5. This pH regulation prevents linoleic acid from binding to the oat material, which appears as an advantageous effect on the isomerization reaction. It is important that the pH decrease in the isomerization mixture is caused by the oat material itself and not by fermentation. Thus the isomerization reaction does not require conventional fermentation, i.e. acidification, but it involves bioconversion. Most of the CLA formed in the isomerization reaction is cis-9,t-11 isomer.

The linoleic acid released according to the invention (from oat) is used in the CLA production. The isomerization reaction can be carried out chemically, enzymatically or microbiologically, for example. The conversion of linoleic acid into CLA is preferably carried out microbiologically. In bioconversion, any bacterium that has the ability of converting linoleic acid into CLA may be used, such as the bacteria mentioned above in the description of the background art. However, isomerization is preferably carried out by means of beneficial bacteria suitable for use in foodstuffs applications, in particular by means of propionic acid bacteria. Strains belonging to the species Propionibacterium freudenreichii, and in particular strains belonging to the subspecies P. freudenreichii ssp. freudenreichii and P. freudenreichii ssp. shermanii are suitable, for example.

Isomerization is carried out in a manner known per se. The components and conditions of the isomerization mixture are selected according to the requirements of the strain to be used so as to obtain an optimal CLA yield. After the publication of the present invention, selection of suitable reaction parameters will be part of the know-how of a person skilled in the art.

The fat hydrolysis and isomerization steps can be carried out in parallel, i.e. simultaneously, or consecutively in different vessels or in the same vessel. Simultaneous performance of the steps in one vessel is considered to be an advantageous alternative due to the ease of the process.

In a particularly preferred embodiment, beneficial bacteria, preferably propionic acid bacteria are added to the ground oat, in which case the linoleic acid released in the lipolysis is directly reacted with the beneficial bacteria, which isomerize the linoleic acid into conjugated linoleic acid. By adjusting the process conditions to suit the lipolysis and isomerization reaction, formation of CLA in considerable amounts in the mixture can be obtained. Water or another suitable medium, particularly a liquid medium, can be used to facilitate mixing. In connection with the present invention, water, for example, has been used as the medium so that the dry solids content of the oat mixture is 5%, in which case a CLA formation of about 1% of the oat dry solids and of about 10% of the oat fat has been obtained.

By combining grain properties with the use of a bacterium capable of isomerization as a catalyst in the isomerization reaction, two of the greatest problems related to the CLA production can be avoided: toxicity of linoleic acid and its poor solubility in water. The ability of a CLA producing strain is preferably combined with a material which contains linoleic acid and lipase and which in the ground form provides linoleic acid for the CLA production without any other additions. Such a material among grain is oat. When materials with no lipase activity, such as wheat, are used, external lipase activity can be added or formed through malting. According to the present invention, the use of detergents needed to “dissolve” external linoleic acid or other harmful additives can be avoided.

The CLA containing (oat) bacteria mixture prepared according to the invention can be used as such, it can be added and used in the preparation of food products and similar functional products, and various CLA containing fractions can be isolated therefrom. The CLA formation can also occur simultaneously with the preparation of a food product. When different products are formed, functional properties of CLA, beneficial bacteria and/or grain, such as oat, can be utilized in the products in a desired manner.

Embodiments where conjugated linoleic acid is isolated from the isomerization mixture are considered preferred embodiments. When the functional effects of both conjugated linoleic acid and bacterial cells are to be utilized, they can be recovered together, concentrated and possibly dried or lyophilized. When water is used as the medium, CLA can be bound to the (oat) bacterial solids by decreasing the pH. According to the invention, conjugated. Linoleic acid can be bound to the solids by adjusting the pH of the reaction mixture to about 3 to 9, preferably to a value below 7.0, most preferably to about 4 to 6.

In the present document, the term food is used in a broad sense covering all edible products which can be in solid, gelled or liquid form, and covering both ready-to-eat products and products to which the product of the invention is added in connection with consumption, as an additive or to be a constituent component of the product. For instance, foods can be products of dairy industry, meat processing industry, food processing industry, beverage industry, bakery industry, confectionery industry and feed industry. Typical products include milk and milk products, such as yoghurt, curdled milk, curd cheese, sour milk, buttermilk and other fermented milk beverages, unripened cheeses and ripened cheeses, snack fillings, etc., beverages, such as whey beverages, fruit beverages, beers and soups. Products of the feed industry constitute another important group.

Preferred applications include lyophilized products, such as CLA and oat containing propionic acid bacterium capsules and powders, and products whose CLA content has been increased utilizing the activity of the propionic acid bacterium. Products including both CLA and oat components, e.g. β-glucan, are particularly preferable, i.e. products expressing the functional effects of both ingredients. An important additional advantage of the present invention is that conjugated linoleic acid can be formed in oat products, and thus the nutritional value of oat can be increased.

The invention will be described in greater detail by means of the following examples. These examples are only intended to illustrate the invention, not to restrict its scope in any way.

### REFERENCE EXAMPLE 1

CLA Concentration of a Product Based on Fermented Oat Bran

The fatty acid content and the concentrations of oleic acid, linoleic acid and CLA of the fermented products described in Finnish patent 88856 were determined as follows. Samples were taken from commercial products, Yosa wild berry and Yosa plum, produced by Bioferme Oy, Finland, and fat was isolated from them by direct saponification and diethyl ether hexane extraction. Methyl esters of fatty acids were prepared by a process catalyzed by sulphuric acid. Table A shows the fatty acid content of the samples in per cents (%) of the total amount of fatty acids, and Table B shows the oleic acid, linoleic acid and CLA (c9,t-11) concentrations as mg/g of sample. Yosa plum and Yosa wild berry products contained hardly any CLA. The very small CLA residues may have resulted from the influence of the analysis methods or they may be isomers of linoleic acid (C_{18:3}) which elute in the gas chromatographic analysis near CLA.

TABLE A
Fatty acid content of Yosa samples, in
percent (%) of the total amount of fatty acids

| | Palmitic | Stearic | Oleic | Linoleic | Linolenic | CLA |
|---|---|---|---|---|---|---|
| | acid | acid | acid | acid | acid | (c-9, t-11) |
| Yosa | C_{16:0} | C_{18:0} | C_{cis-9-18:1} | C_{18:2} | C_{18:3} | C_{18:2} |
| Plum | 16.07 | 1.21 | 32.05 | 39.63 | 2.04 | 0.05 |
| Wild | 15.58 | 1.14 | 30.93 | 39.45 | 3.68 | 0.07 |
| berry | | | | | | |

TABLE B
Oleic acid, linoleic acid and CLA concentrations of
Yosa samples, mg/g of sample

| | Oleic acid | Linoleic acid | CLA (c-9, t-11) |
|---|---|---|---|
| Yosa | mg/g of sample | mg/g of sample | mg/g of sample |
| Plum | 1.77 | 2.15 | 0.003 |
| Wild berry | 1.65 | 2.08 | 0.004 |

### EXAMPLE 1

CLA Production in an Oat/Water Mixture by Means of Propionic Acid Bacteria.

To prove the effect of the process according to the invention, a test was performed where propionic acid bacteria cells were added to an oat/water mixture for use as an isomerization catalyst. Oatmeal produced by grinding untempered oat (variety Lisbeth) through a screen of 0.5 mm was used in the test. A 5% water mixture (w/v) was prepared from the oatmeal and the mixture was homogenized by an Ultra Turrax apparatus for about two minutes.

Two propionic acid bacteria strains were used in the test, i.e. Propionibacterium freudenreichii subsp. shermanii JS (PJS) and Propionibacterium freudenreichii subsp. freudenreichii 131 (P131).

Culturing of PJS cells for the test was carried out as described in Rainio, A., Vahvaselkä, M., Suomalainen, T. ja Laakso, S., Reduction of linoleic acid inhibition in production of conjugated linoleic acid by Propionibacterium freudenreichii ssp. shermanii, Can. J. Microbiol. 47 (2001), 735-740. The P131 strain was cultured in MRS liquor (LabM). The cells were centrifuged (6000 rpm, 20 min) to separate them and elutriated in a small amount of peptone salt solution, which contained 0.1% of bacteriological peptone (LabM), and 0.85% of NaCl. The cell suspension was added to the oatlwater mixture (a′ 100 ml) to obtain a cell concentration of about 1×10¹⁰ cfu/ml. The pH of the mixture was adjusted to 7.0 by 1 M NaOH solution, and the hydrolysis and isomerization reaction was allowed to occur at 25° C. During the first 17 hours, the oat/water mixture was allowed to hydrolyze without pH regulation, as a result of which the pH decreased to approximately 4.8. After this, the pH of the mixture was raised to 8.0 by NaOH solution and the adjustment was repeated approximately every 1.5 to 2 hours for about eight hours. The pH of the mixture thus remained approximately between 7.5 and 8.0. After this, isomerization was continued without pH regulation until the total time was about 40 hours.

By comparison, a test was carried out where a corresponding volume of peptone salt solution was added to the oat/water mixture instead of the PJS or P131 cell suspension. During the first 17 hours, the pH of the mixture decreased to about 5.4. After this, the pH was adjusted as in the test described above.

In the test, release of linoleic acid as a result of the activity of the natural lipase enzyme of oat and isomerization of this free linoleic acid into CLA by means of microbe cells were followed. Samples of 0.5 ml were taken from the oat/water mixture, and these were cold-dried before the fatty acid analysis. The amounts of different fatty acids were determined from the samples by means of gas chromatography. The analysis of fatty acids was carried out as described in Suutari M., Liukkonen, K. and Laakso, S., Temperature adaptation in yeasts: the role of fatty acids, J. Gen. Microbiol. 136 (1990), 1469-1474. The fatty acids included in the samples were identified by comparing their retention times to the retention times of known fatty acid standards. Conjugated linoleic acid was identified utilizing a preparation from Sigma, which was a mixture of cis and trans-9,11 and cis and trans-10,12 isomers of CLA. A methyl ester of C17:0 fatty acid (heptadecanoic acid methyl ester, Sigma) was used as internal standard in the quantification of fatty acids.

Samples of 1.5 ml, which were cold-dried, were taken from the oat/water mixture for lipid class analysis of the fatty acids. The lipid class analysis was carried out as described in Liukkonen, K. H., Montfoort, A. and Laakso, S., Water-induced lipid changes in oat processing, J. Agric. Food Chem. 40 (1992) 126-130.

The amounts of linoleic acid and CLA formed were calculated as a function of reaction time per solids sample. The test was carried out in the oat/water mixture both in the presence of propionic acid bacteria (PAB) and without them. The results are presented in Table 1.

TABLE 1
Amounts of linoleic acid and CLA formed as a function of
reaction time calculated per solids sample.

| | Linoleic acid (mg/g | | | CLA (mg/g of dry | | |
|---|---|---|---|---|---|---|
| | of dry solids) | | | solids) | | |
| Time | PJS | P131 | no PHB | PJS | P131 | no PHB |
| (h) | cells | cells | cells | cells | cells | cells |
| 0 | 41.9 | 36.3 | 43.4 | <0.1 | <0.1 | <0.1 |
| 17 | 40.7 | — | — | 0.6 | — | — |
| 21, 5 | 36.3 | — | — | 4.8 | — | — |
| 25 | 33.6 | — | — | 6.5 | — | — |
| 41 | 32.3 | 32.7 | 42.3 | 7.6 | 0.7 | <0.1 |

The CLA formation thus required the functioning of propionic acid bacterium as isomerization catalyst in the oat/water mixture. When the PJS strain was used, considerable amounts of CLA were formed, 7.6 mg/g of dry solids. This amount was 7.3% of the fat included in oat.

Table 2 shows the distribution of linoleic acid and CLA into different lipid classes when the oat/water mixture was incubated together with PJS cells. PL=polar lipids, TG=triglycerides, DG=diglycerides and FFA=free fatty acids.

TABLE 2
Distribution of linoleic acid and CLA into different
lipid classes (%) during the test.

| Time | Compound | PL | TG | DG | FFA |
|---|---|---|---|---|---|
| 0 h | linoleic acid | 12 | 81 | 3 | 4 |
| 17 h | linoleic acid | 9 | 49 | 5 | 37 |
| 41 h | linoleic acid | 12 | 40 | 5 | 43 |
| 41 h | CLA | 4 | 8 | 1 | 87 |

The results show that at the beginning of the test, most of the linoleic acid was bound to triglycerides and only 4% of it was in the form of free fatty acid. However, after a 17-hour hydrolysis, nearly 40% of the linoleic acid had been released from the triglycerides. The forming CLA was mostly (nearly 90%) in the form of free fatty acid. At least 80% of the CLA formed was cis-9,trans-11 isomer.

The concentrations of living propionic acid bacterial cells were determined using buffered sodium lactate agar, which contained 0.5% of tryptone (LabM), 1% of yeast extract (LabM), 16.8 mUl of 50% Na lactate (Merck), 1% of disodium salt of P-glycerophosphate (Merck) and 1.5% of agar (LabM). The plates were incubated anaerobically at 30° C. for 6 days. At the beginning of the test, the PJS concentration was 9.0×10⁹ cfu/ml and after 20 hours 7.5×10⁹ cfu/ml. On the basis of the results, the propionic acid bacterial cells thus did not grow in the oat/water mixture during the reaction.

The pH of the oat/water mixture tended to decrease rapidly regardless whether propionic acid bacterium had been added to the mixture or not. The pH decrease is caused by dissolution of acid components of oat in water. The process does not thus require that the cells be able to ferment oat, whereby the organic acids formed would decrease the pH.

### EXAMPLE 2

CLA Production from Other Grain Species by Propionic Acid Bacteria.

Example 1 was repeated using barley and rye instead of oat. Propionibacterium freudenreichii subsp. shermanii JS (PJS) cells were used as the propionic acid bacterium. On the basis of the results, CLA production in a mixture of barley or rye in water was considerably weaker than in the oat/water mixture; 0.91 mg of CLA / g of dry solids was formed in barley and 0.83 mg in rye during a 41-hour incubation where the pH was adjusted to 8.0 between 17 and 25 hours. The poorer results are partly explained by the fact that the linoleic acid concentration of these grain materials is smaller than that of oat. Furthermore, it is known that they do not include lipase activity without germination. However, based on the results, it is clear that the process according to the invention also functions in other grain materials. Formation of free linoleic acid can be enhanced by adding external lipase activity to the reaction mixture, in which case the process yield can be improved significantly from the values given above.

### EXAMPLE 3

Effect of pH on CLA Formation.

The effect of pH of the oat/water mixture on the CLA formation was analyzed by the following tests:
the pH was not adjusted at all
the pH was adjusted to 8.0 between 0 to 8 hours (thus the test did not include a separate fat hydrolysis step)
the pH was adjusted to 7.0 between 17 to 25 hours
the pH was adjusted to 8.0 between 17 and 25 hours
the pH was adjusted to 8.5 between 17 to 25 hours
the pH was adjusted to 9.0 between 17 and 25 hours.

In all the above-mentioned tests, the PJS bacteria strain was added to the oat/water mixture as described in example 1. The other test arrangements were also the same.

In addition, a test was performed in a fermenter, where the pH of the oat/water mixture was kept at 8.5 during the whole isomerization step (between 17 and 41 h) by means of automatic addition of NaOH solution. In the 17-hour lipolysis step preceding it, the pH decreased to 4.7. The volume of the oat/water mixture was 1.5 litres, temperature 25° C. and mixing speed 100 rpm. The concentration of living PJS cells was 1.1×10¹⁰ cfu/ml at the beginning of the test and 8.4×10⁹ cfu/ml at the end of the test.

The results are shown in Table 3. According to the results, the CLA formation was effective when the pH of the oat/water mixture was adjusted to between 8.0 and 8.5 after the lipase enzyme had released linoleic acid. This proceeded best at a pH lower than that of the isomerization reaction. The fastest and greatest CLA formation was achieved when the pH of the oat/water mixture was kept at 8.5 by continuous regulation during the whole isomerization step. This reflects the importance of even pH level suitable for the isomerization reaction to the process.

TABLE 3
Influence of the pH of the oat/water mixture
on the CLA formation.

CLA (mg/g
of dry
solids)

| | 25 h | 41 h |
|---|---|---|
| pH not adjusted | | 0.9 |
| pH adusted to a approx. 8.0 between 0-8 h | 3.5 | |
| PH adjusted to approx. 7.0 between 17-25 h | | 3.9 |
| PH adjusted to approx. 8.0 between 17-25 h | 6.5 | 7.6 |
| pH adjusted to approx. 8.5 between 17-25 h | | 7.9 |
| pH adjusted to approx. 9.0 between 17-25 h | | 6.0 |
| pH kept at 8.5 by automatic regulation | 8.2 | 9.3 |
| between 17-41 h | | |

### EXAMPLE 4

Concentration of Produced CLA into Oat Dry Solids by Means of pH Decrease.

CLA production in an oat/water mixture was performed according to example 1 by means of PJS cells. After this, the pH of the oat/water mixture was adjusted to 8.0 by NaOH solution or to 4.5 by HCl solution. The mixtures were centrifuged and CLA concentrations were determined from supernatants and oat bacteria masses. The CLA distribution was as follows: at the pH of 8.0, 85% of the CLA was in the solids and 15% in the liquid step, at the pH of 4.5, 100% of CLA was in the solids. The result provides a process by means of which, utilizing the pH decrease, CLA can be made to concentrate into the solids formed by oat and bacterial cells, and thus the CLA is not removed together with the medium.

1. A process for preparing conjugated linoleic acid by microorganisms, characterized by hydrolyzing oat fat and isomerizing the linoleic acid released in the hydrolysis into conjugated linoleic acid by the microorganisms.

2. A process according to claim 1, characterized in that the grain is untreated oat, pretreated oat or an oat fraction.

3. A process according to claim 1, characterized in that the fat hydrolysis is caused by the enzyme activity of oat.

4. A process according to claim 1, characterized in that the fat hydrolysis is carried out by adding external enzyme activity.

5. A process according to claim 1, characterized in that isomerization is carried out by a beneficial bacterium (bacteria).

6. A process according to claim 1, characterized in that the beneficial bacterium is a propionic acid bacterium.

7. A process according to claim 6, characterized in that the propionic acid bacterium is a strain belonging to the species Propionibacterium freudenreichii, preferably a strain belonging to its subspecies Propionibacterium freudenreichii ssp. freudenreichii or Propionibacterium freudenreichii ssp. shermanii.

8. A process according to claim 7, characterized in that the propionic acid bacterium is Propionibacterium freudenreichii ssp. shermanii JS, DSM 7067.

9. A process according to claim 1, characterized in that isomerization is carried out at a pH of about 6.5 to 9.5.

10. A process according to claim 9, characterized in that isomerization is preferably carried out at a pH of about 7.0 to 9.0, more preferably at a pH of about 8.0 to 8.5.

11. A process according to claim 1, characterized in that the hydrolysis and isomerization steps are carried out consecutively.

12. A process according to claim 1, characterized in that the hydrolysis and isomerization steps are carried out in parallel.

13. A process according to claim 1, characterized in that the preparation of conjugated linoleic acid occurs in connection with the preparation of a food product.

14. A process according to claim 1, characterized in that mainly cis-9, trans-11 isomer of conjugated linoleic acid is formed therein.

15. A process according to claim 1, characterized in that conjugated linoleic acid is fixed to solids by adjusting the pH of the reaction mixture to about 3 to 9, preferably to a value lower than 7.0, most preferably to about 4 to 6.

16. A process according to claim 1, characterized in that conjugated linoleic acid is isolated from the reaction broth and possibly dried.

17. A process according to claim 1, characterized in that conjugated linoleic acid, bacterial cells and the oat material used as starting material, which is preferably oat material are concentrated and possibly dried.

18. A process according to claim 17, characterized in that linoleic acid, bacterial cells and oat material used as the starting material are recovered, concentrated and lyophilized.

19. Oat for use in the preparation of conjugated linoleic acid.

20. A process for preparing conjugated linoleic acid from linoleic acid, characterized in that oat is used as the source of linoleic acid.
